(19) United States
(12) Patent Application Publication
(10) Pub. No.: US 2023/0293317 A1
(43) Pub. Date: Sep. 21, 2023

(54) HAPTIC PERCEPTION SYSTEM WITH STEREOGNOSTIC AND PROPRIOCEPTIVE SENSES INDUCTION FOR AN ARTIFICIAL LIMB OR A SENSORY DISRUPTED LIMB

(71) Applicants: Ali Moradi, Mashhad (IR); Mahla Daliri, Mashhad (IR); Alireza Akbarzadeh, Mashhad (IR); Mohammadreza Akbarzadeh, Mashhad (IR); Hamed Rafiei, Mashhad (IR); Mona Meibodi, Mashhad (IR)

(72) Inventors: Ali Moradi, Mashhad (IR); Mahla Daliri, Mashhad (IR); Alireza Akbarzadeh, Mashhad (IR); Mohammadreza Akbarzadeh, Mashhad (IR); Hamed Rafiei, Mashhad (IR); Mona Meibodi, Mashhad (IR)

(73) Assignees: Ali Moradi, Mashhad (IR); Alireza Akbarzadeh, Mashhad (IR); Mohammadreza Akbarzadeh, Mashhad (IR)

(21) Appl. No.: 18/322,612

(22) Filed: May 24, 2023

Related U.S. Application Data
(63) Continuation-in-part of application No. PCT/IB2021/060932, filed on Nov. 24, 2021.
(60) Provisional application No. 63/117,475, filed on Nov. 24, 2020.

(51) Int. Cl. A61F 2/58; A61F 2/70

(52) U.S. Cl. CPC A61F 2/583; A61F 2/70; A61F 2002/704

(57) ABSTRACT

A haptic perception system for a hand prosthesis. The haptic perception system includes a stereognosis unit, a proprioception unit, and one or more processors. The stereognosis unit includes a plurality of pressure sensors attached onto an external surface of the hand prosthesis, a plurality of passive magnetic tags embedded in a hypoderm tissue of a residual limb of the amputee, and a plurality of electromagnetic coils attached onto a skin of the residual limb of the amputee. When a first electromagnetic coil is activated, a first passive magnetic tag moves toward the first electromagnetic coil and, to thereby, stimulates pressure sensory receptors present in the hypoderm tissue of the residual limb.

### CROSS-REFERENCE TO RELATED APPLICATION

This application is a continuation-in part of International Application No. PCT/IB2021/060932, filed Nov. 24, 2021, and entitled “HAPTIC PERCEPTION SYSTEM WITH STEREOGNOSTIC AND PROPRIOCEPTIVE SENSES INDUCTION FOR AN ARTIFICIAL LIMB OR A SENSORY DISRUPTED LIMB” which claims the benefit of priority from U.S. Provisional Pat. Application Serial No. 63/117,475, filed on Nov. 24, 2020, and entitled “MERGE THE PROSTHESIS WITH AMPUTEES BODY, PROVIDING SENSORY FEEDBACK BY APPLYING MAGNETISM” which are both incorporated herein by reference in their entirety.

### TECHNICAL FIELD

The present disclosure generally relates to artificial limb technology and, more particularly, relates to stereognostic and proprioceptive senses induction in a limb prosthesis and/or a sensory disrupted limb by applying magnetism.

### BACKGROUND

Stereognosis is an ability to perceive a shape and form of solid objects, and therefore its identity, with skin tactile sensory receptor stimulation in the absence of visual and auditory stimuli. Two-point discrimination may be the smallest separation distance at which two points applied simultaneously to the skin can be distinguished as two distinct points, not one. It is, in fact, a measurement of innervation density ranges from 2 mm in the finger pads to 40 mm on the back. Human hand has got a developed complicated sensory system and its function strongly depends on this system. In other words, hands without sensory system may be considered as hands without function.

Proprioception is the sense of self-movement and body position in space that may allow the body to perform simultaneous actions. The system may continuously monitor the posture and adjust muscle activity as needed. It may be mediated by two main types of proprioceptors consisting of muscle spindles, which may be embedded in skeletal muscle fibers and Golgi organs, which may lie at the interface of muscles and tendons, proprioception plays an important role in providing stability besides the visual and vestibular organ senses (balance triangle). A significant proprioception decline and, therefore, disrupted dynamic balance, with age is reported in literature.

A variety of medical conditions may be associated with absence or a disrupted sensory system which may include limb amputation, neurologic conditions like multiple sclerosis (MS), peripheral neural injuries caused by trauma or prolonged usage of vibratory devices, peripheral neuropathies caused by diabetes, alcohol abuse, and renal dysfunction. For example, median nerve injury in volar aspect of wrist may cause sensory loss in a significant area of hand and surgical neural repair cannot retrieve the nerve function completely because of growing nerve fiber misorientation.

Besides, nerve function partial recovery and healing may need at least a period of six months during which time period, a patient may have a significant trouble with his/her hand movements. Many myoelectric prosthesis users continue to have complaints about malfunctioning aspects of their prosthesis systems mainly because of sensory perception absence so that the user may not have a precise control over gripping motions, especially while attempting to conduct delicate movements. There is, therefore, a need for an alternative sensory system by which stereognosis and proprioception are induced in an artificial or sensory disrupted limb.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a haptic perception system for a hand prosthesis of an amputee. The haptic perception system may include a stereognosis unit, a proprioception unit, and one or more processors. In an exemplary embodiment, the stereognosis unit may include a plurality of pressure sensors attached onto an external surface of the hand prosthesis. In an exemplary embodiment, a first pressure sensor from the plurality of pressure sensors may be configured to measure a pressure applied to the first pressure sensor.

In an exemplary embodiment, the stereognosis unit may further include a plurality of passive magnetic tags. In an exemplary embodiment, the plurality of passive magnetic tags may be configured to be embedded in a hypoderm tissue of a residual limb of the amputee. In an exemplary embodiment, a first passive magnetic tag from the plurality of passive magnetic tags may be associated with the first pressure sensor. In an exemplary embodiment, the haptic perception system may further include a plurality of electromagnetic coils. In an exemplary embodiment, the plurality of electromagnetic coils may be configured to be attached onto a skin of the residual limb of the amputee. In an exemplary embodiment, a first electromagnetic coil from the plurality of electromagnetic coils may be associated with the first pressure sensor. In an exemplary embodiment, the first electromagnetic coil may be configured to be placed above the first passive magnetic tag from the plurality of passive magnetic tags. In an exemplary embodiment, the first electromagnetic coil may be configured to be placed exactly face to face the first passive magnetic tag from the plurality of passive magnetic tags. In an exemplary embodiment, the first electromagnetic coil may be configured to urge the first passive magnetic tag to move toward the first electromagnetic coil responsive to activation of the first electromagnetic coil.

In an exemplary embodiment, the one or more processors may be configured to capture a first set of data from the first pressure sensor. In an exemplary embodiment, the first set of data may be associated with a pressure applied to the first pressure sensor. In an exemplary embodiment, the one or more processors may further be configured to detect the pressure applied to the first pressure sensor being higher than a threshold based on the first set of data.

In an exemplary embodiment, the one or more processors may also be configured to activate the first electromagnetic coil by sending a first activation signal to the first electromagnetic coil responsive to the pressure applied to the pressure sensor being higher than the threshold. In an exemplary embodiment, when the first electromagnetic coil is activated, the first passive magnetic tag may be configured to simulate pressure sensory receptors present in the hypoderm tissue of the residual limb by the first passive magnetic tag moving toward the first electromagnetic coil. In an exemplary embodiment, pressure sensory receptors may refer to Meissner’s and Pacinian corpuscles.

In an exemplary embodiment, the proprioception unit may include a strain gauge attached to a finger of the hand prosthesis. In an exemplary embodiment, the strain gauge may be configured to measure strain in the finger of the hand prosthesis. In an exemplary embodiment, the proprioception unit may include a muscle magnetic tag. In an exemplary embodiment, the muscle magnetic tag maya be configured to be embedded in a muscle of the residual limb. In an exemplary embodiment, the muscle magnetic tag may be associated with the strain gauge.

In an exemplary embodiment, the proprioception unit may further include a row of electromagnetic coils. In an exemplary embodiment, the row of electromagnetic coils may be configured to be attached onto the skin of the residual limb of the amputee. In an exemplary embodiment, the row of electromagnetic coils may be configured to be arranged along a first axis. In an exemplary embodiment, the first axis may be parallel to a main longitudinal axis of a tendon of the residual limb of the amputee. In an exemplary embodiment, the tendon of the residual limb of the amputee may directly be connected to the muscle of the residual limb. In an exemplary embodiment, each electromagnetic coil from the row of electromagnetic coils may be configured to urge the muscle magnetic tag to move toward the each electromagnetic coil responsive to activation of the each electromagnetic coil.

In an exemplary embodiment, the one or more processors may be configured to capture a second set of data from the strain gauge. In an exemplary embodiment, the second set of data associated with the strain of the finger of the hand prosthesis. In an exemplary embodiment, the one or more processors may further be configured to activate one electromagnetic coil from the row of electromagnetic coils by sending a second activation signal to the one electromagnetic coil from the row of electromagnetic coils based on the second set of data.

In an exemplary embodiment, each pressure sensor from the plurality of pressure sensors may be configured to measure a pressure applied to the each pressure sensor. In an exemplary embodiment, each passive magnetic tag from the plurality of passive magnetic tags may be associated with a respective pressure sensor from the plurality of pressure sensors. In an exemplary embodiment, each electromagnetic coil from the plurality of electromagnetic coils may be associated with a respective pressure sensor from the plurality of pressure sensors.

In an exemplary embodiment, each electromagnetic coil from the plurality of electromagnetic coils may be configured to be placed above a respective passive magnetic tag from the plurality of passive magnetic tags. In an exemplary embodiment, each electromagnetic coil from the plurality of electromagnetic coils may be configured to urge the respective passive magnetic tag to move toward the each electromagnetic coil responsive to activation of the each electromagnetic coil. In an exemplary embodiment, each electromagnetic coil from the plurality of electromagnetic coils may be configured to urge the respective passive magnetic tag to move away from the each electromagnetic coil responsive to activation of the each electromagnetic coil.

In an exemplary embodiment, the first electromagnetic coil may be attached onto a hollow base. In an exemplary embodiment, the hollow base may be configured to be attached onto the skin of the residual limb. In an exemplary embodiment, the hollow base may be configured to prevent a direct contact between the first electromagnetic oil and the skin of the residual limb.

In an exemplary embodiment, the first passive magnetic tag may be embedded in a capsule. In an exemplary embodiment, the capsule may be covered by a hydroxyapatite layer. In an exemplary embodiment, a distance between two adjacent passive magnetic tags of the plurality of passive magnetic tags may be less than 7 mm. In an exemplary embodiment, a distance between two adjacent electromagnetic coils of the plurality of electromagnetic coils is less than 7 mm. In an exemplary embodiment, the row of electromagnetic coils may include four electromagnetic coils including a second electromagnetic coil, a third electromagnetic coil, a fourth electromagnetic coil, and a fifth electromagnetic coil.

In an exemplary embodiment, the one or more processors may further be configured to activate the second electromagnetic coil from the row of electromagnetic coils by sending the second activation signal to the second electromagnetic coil from the row of electromagnetic coils responsive to the strain of the finger of the hand prosthesis being less than a first predetermined amount.

In an exemplary embodiment, the one or more processors may further be configured to activate the third electromagnetic coil from the row of electromagnetic coils by sending the second activation signal to the third electromagnetic coil from the row of electromagnetic coils responsive to the strain of the finger of the hand prosthesis being greater than the first predetermined amount and less than a second predetermined amount.

In an exemplary embodiment, the one or more processors may further be configured to activate the fourth electromagnetic coil from the row of electromagnetic coils by sending the second activation signal to the fourth electromagnetic coil from the row of electromagnetic coils responsive to the strain of the finger of the hand prosthesis being greater than the second predetermined amount and less than a third predetermined amount.

In an exemplary embodiment, the one or more processors may further be configured to activate the fifth electromagnetic coil from the row of electromagnetic coils by sending the second activation signal to the fifth electromagnetic coil from the row of electromagnetic coils responsive to the strain of the finger of the hand prosthesis being greater than the third predetermined amount and less than a fourth predetermined amount.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a haptic perception system with a hand prosthesis of an amputee, consistent with one or more exemplary embodiments of the present disclosure.

FIG. 1B illustrates a haptic perception system with a hand prosthesis of an amputee, consistent with one or more exemplary embodiments of the present disclosure.

FIG. 2 illustrates a first electromagnetic coil and a first passive magnetic tag of a stereognosis unit, consistent with one or more exemplary embodiments of the present disclosure.

FIG. 3A illustrates a proprioception unit of a haptic perception system with a hand prosthesis of an amputee, consistent with one or more exemplary embodiments of the present disclosure.

FIG. 3B illustrates a row of electromagnetic coils and a magnet of a proprioception unit, in a scenario in which a second electromagnetic coil is activated, consistent with one or more exemplary embodiments of the present disclosure.

FIG. 3C illustrates a row of electromagnetic coils and a magnet, in a scenario in which a third electromagnetic coil is activated, consistent with one or more exemplary embodiments of the present disclosure.

FIG. 3D illustrates a row of electromagnetic coils and a magnet, in a scenario in which a fourth electromagnetic coil is activated, consistent with one or more exemplary embodiments of the present disclosure.

FIG. 4 illustrates an exemplary embodiment of a processing unit in which an exemplary embodiment of the present disclosure, or portions thereof, may be implemented as computer-readable code, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

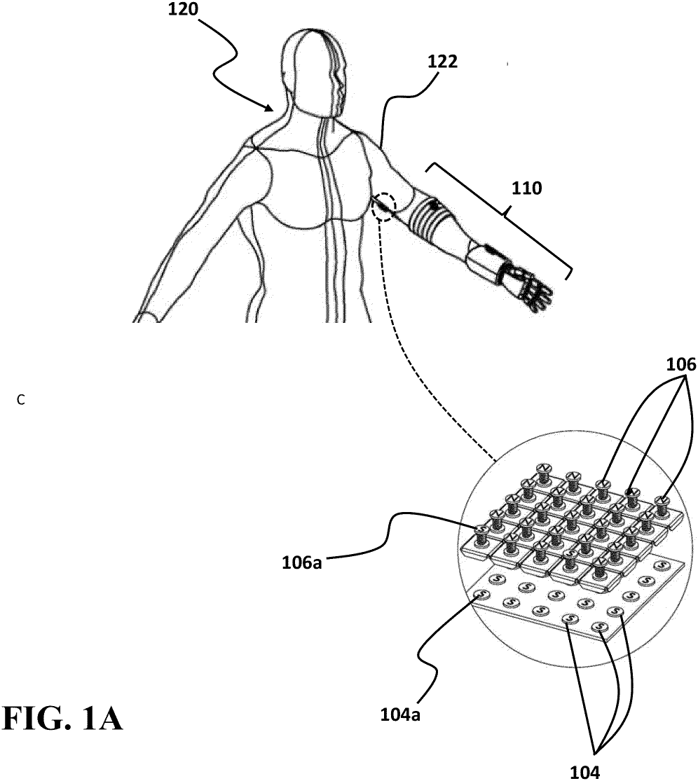

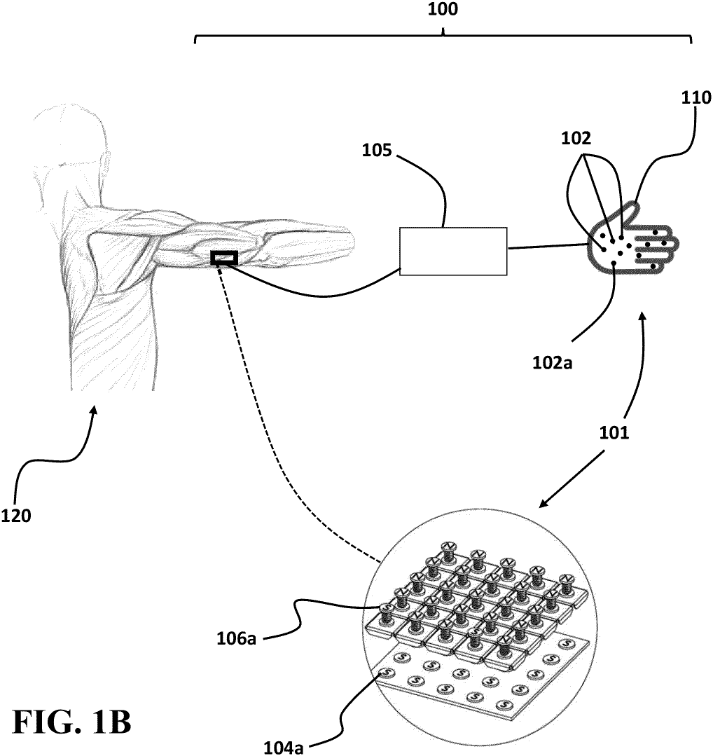

The present disclosure is directed to exemplary embodiments of a haptic perception system. An exemplary haptic perception system may induce stereognostic and proprioceptive senses in a limb prosthesis or a sensory disrupted limb by applying magnetism. FIG. 1A shows a haptic perception system with a hand prosthesis 110 of an amputee 120, consistent with one or more exemplary embodiments of the present disclosure. FIG. 1B shows a haptic perception system 100 with a hand prosthesis 110 of an amputee 120, consistent with one or more exemplary embodiments of the present disclosure. In an exemplary embodiment, an exemplary haptic perception system may be used to induce stereognostic and proprioceptive senses in an exemplary hand prosthesis such as hand prosthesis 110. In an exemplary embodiment, the haptic perception system 100 may include a stereognosis unit 101 and a proprioception unit 103.

In an exemplary embodiment, stereognosis unit 101 may include a plurality of pressure sensors 102. In an exemplary embodiment, plurality of pressure sensors 102 may be attached onto an external surface of hand prosthesis 110. In an exemplary embodiment, a first pressure sensor 102a from plurality of pressure sensors 102 may measure a pressure applied to first pressure sensor 102a. In an exemplary embodiment, a pressure sensor may be designed to measure or monitor tactile pressure. It may be based on passive radiofrequency identification (RFID) sensor tags. In an exemplary embodiment, a pressure sensor may react to a pressure change causing the passive tag to generate an electromagnetic field. The pressure sensor/passive tag may react to the electromagnetic field and respond by sending a signal to an interrogator. The interrogator receives the reflected signal, measures the returned signal strength indications (“RSSI”) of the reflected signal and sends the RSSI measurements and identification of the responding RFID sensors to the processor to determine the pressure. In an exemplary embodiment, stereognosis unit 101 may further include a plurality of passive magnetic tags 104. In an exemplary embodiment, plurality of passive magnetic tags 104 may be embedded in a hypoderm tissue of a residual limb 122 of amputee 120. In an exemplary embodiment, each of plurality of passive magnetic tags 104 may include a Radio-frequency identification (RFID) tag coated with a biocompatible silicon or any other biocompatible material. In an exemplary embodiment, plurality of passive magnetic tags 104 may be embedded under the skin in hypoderm where the number of Meissner’s and Pacinian receptors are maximum. In an exemplary embodiment, plurality of passive magnetic tags 104 may be embedded in a hypoderm tissue of a residual limb 122 of amputee 120 by a surgeon through a surgery. In an exemplary embodiment, residual limb 122 of amputee 122 may refer to an arm of amputee 120 or a forearm of amputee 120.

In an exemplary embodiment, stereognosis unit 101 may further include a plurality of electromagnetic coils 106. In an exemplary embodiment, plurality of electromagnetic coils 106 may be attached onto a skin 204 of residual limb 122 of amputee 120. In an exemplary embodiment, as shown in FIG. 1A, in an exemplary embodiment, plurality of electromagnetic coils 106 may be arranged in a matrix arrangement. In an exemplary embodiment, a distance between two adjacent electromagnetic coils from plurality of electromagnetic coils 106 may be larger than a predetermined distance. In an exemplary embodiment, the predetermined distance may be 7 mm. In an exemplary embodiment, skin 204 of residual limb 122 of amputee 120 may refer to a skin 204 of the arm or forearm of amputee 120. In an exemplary embodiment, first electromagnetic coil 106a may be placed above first passive magnetic tag 104a. In an exemplary embodiment, first passive magnetic tag 104a may be placed at an inner side 242 of skin 204 of amputee 120 and first electromagnetic coil 106a may be placed at an outer side 244 of skin 204 of amputee 120. In an exemplary embodiment, inner side 242 of skin 204 of amputee 120 may refer to a side of skin 204 at which the subcutaneous tissue of the skin of amputee 120 is present. In an exemplary embodiment, outer side 244 of skin 204 of amputee 120 may refer to a side of skin 204 at which the epidermis of skin 204 of amputee 120 is present. In an exemplary embodiment, when first electromagnetic coil 106a is placed above first passive magnetic tag 104a, it may mean that first electromagnetic coil 106a is placed onto outer side 244 of skin 204 of amputee 120 and first passive magnetic tag 104a is placed onto inner side 242 of skin 204 of amputee 120 in such a way that a hypothetical line between a center of mass of first electromagnetic coil 106a and a center of mass of first passive magnetic tag 104a is perpendicular to a main plane of skin 204 of amputee 120. In an exemplary embodiment, the main plane of skin 204 may refer to a plane that is tangential to epidermis of skin 204 at a place that first electromagnetic coil 106a is in contact with epidermis of skin 204. . In an exemplary embodiment, first electromagnetic coil 106a and first passive magnetic tag 104a may be placed at two opposite sides of skin 204 of amputee 120 in such a way that first electromagnetic coil 106a and first passive magnetic tag 104a are placed in front of each other and at the two opposite sides of skin 204 of amputee 120.

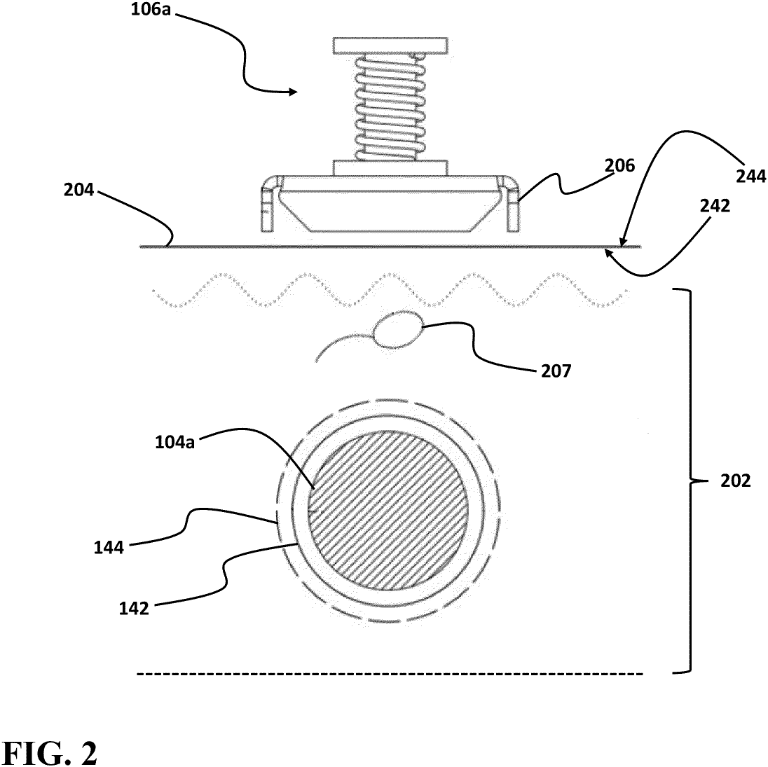

FIG. 2 shows first electromagnetic coil 106a and first passive magnetic tag 104a of stereognosis unit 101, consistent with one or more exemplary embodiments of the present disclosure. As shown in FIG. 2, in an exemplary embodiment, first passive magnetic tag 104a may be inserted into a hypoderm tissue 202 of residual limb 122 of amputee 120. As shown in FIG. 2, in an exemplary embodiment, first passive magnetic tag 104a may be embedded in a capsule 142 which may be covered by a hydroxyapatite layer 144. In an exemplary embodiment, capsule 142 may be made of a biocompatible material, such as titanium. In an exemplary embodiment, hydroxyapatite layer 144 may stabilize first passive magnetic tag 104a in the desired spot (at musculotendinous junction) by organizing fibrous tissue around. In an exemplary embodiment, first electromagnetic coil 106a may be attached onto skin 204 of residual limb 122 of amputee 120. As shown in FIG. 2, in an exemplary embodiment, first electromagnetic coil 106a may be attached onto skin 204 of residual limb 122 of amputee 120 in such a way that first electromagnetic coil 106a is placed above first passive magnetic tag 104a. As further shown in FIG. 2, in an exemplary embodiment, first electromagnetic coil 106a may be attached onto a hollow base 206 and hollow base 206 may be attached onto skin 204 of residual limb 122 of amputee 120. In an exemplary embodiment, hollow base 206 may prevent direct contact between first electromagnetic coil 106a and skin 204 of residual limb 122 of amputee 120.

In an exemplary embodiment, when first electromagnetic coil 106a is activated, first electromagnetic coil 106a may urge first passive magnetic tag 104a to move toward first electromagnetic coil 106a. In an exemplary embodiment, when first electromagnetic coil 106a is activated, a magnetic field may be created around first electromagnetic coil 106a that may urge first passive magnetic tag 104a to move toward first electromagnetic coil 106a. In an exemplary embodiment, when first passive magnetic tag 104a moves toward first electromagnetic coil 106a, first passive magnetic tag 104a may stimulate pressure sensory receptors 207. For purpose of reference, it should be understood that, pressure sensory receptors are naturally present in the hypoderm tissue and under the skin of a human body. It may also be understood that when pressure sensory receptors are stimulated, stereognostic sense may be induced and transferred to the brain. In an exemplary embodiment, first electromagnetic coil 106a may urge first passive magnetic tag 104a to move away from first electromagnetic coil 106a and, to thereby, may stimulate pressure sensory receptors 207. In an exemplary embodiment, pressure sensory receptors may refer to Meissner’s and/or Pacinian corpuscles.

In an exemplary embodiment, an exemplary haptic perception system may further include one or more processors 105. In an exemplary embodiment, one or more processors 105 may be configured to capture a first set of data from first pressure sensor 102a. In an exemplary embodiment, one or more processors 105 may further be configured to detect that the pressure applied to first pressure sensor 102a is higher than a threshold based on the first set of data. For example, the threshold may be 0.01 Newton. In an exemplary embodiment, when the pressure applied to first pressure sensor 102a is higher than 0.01 Newton, one or more processors 105 may receive data associated with the pressure applied to first pressure sensor 102a and then detect that the pressure applied to first pressure sensor 102a is higher than 0.01 Newton. In an exemplary embodiment, one or more processors 105 may further be configured to activate first electromagnetic coil 106a by sending a first activation signal to first electromagnetic coil 106a when the pressure applied to first pressure sensor 102a is higher than the threshold.

In an exemplary embodiment, plurality of passive magnetic tags 104 may include more passive magnetic tags in addition to first passive magnetic tag 104a. In an exemplary embodiment, as shown in FIG. 1A, plurality of passive magnetic tags 104 may be arranged in a matrix arrangement. In an exemplary embodiment, a distance between two adjacent passive magnetic tags from plurality of passive magnetic tags 104 may be larger than the predetermined distance. In an exemplary embodiment, plurality of passive magnetic tags 104 may be attached onto a plate and then the plate may be embedded into hypoderm tissue 202 of residual limb 122 of amputee 120. In an exemplary embodiment, all of plurality of passive magnetic tags 104 may be similar in structure and functionality to first passive magnetic tag 104a. In an exemplary embodiment, plurality of electromagnetic coils 106 may include more electromagnetic coils in addition to first electromagnetic coil 106a. In an exemplary embodiment, a number of plurality of passive magnetic tags 104 may be the same as a number of plurality of electromagnetic coils 106. In an exemplary embodiment, each respective electromagnetic coil from plurality of electromagnetic coils 106 may be placed above the respective passive magnetic tag from plurality of passive magnetic tags 104.

In an exemplary embodiment, plurality of pressure sensors 102 may include more pressure sensors in addition to first pressure sensor 102a. In an exemplary embodiment, all pressure sensors of plurality of pressure sensors 102 may be similar in structure and functionality to first pressure sensor 102a. In an exemplary embodiment, a number of plurality of pressure sensors 102 may be the same as the number of plurality of electromagnetic coils 106. In an exemplary embodiment, when one or more processors 105 detects that the pressure applied to a pressure sensor from plurality of pressure sensors 102 is higher than the threshold, one or more processors 105 may send the first activation signal to the respective electromagnetic coil from plurality of electromagnetic coils 106. In an exemplary embodiment, when, the respective electromagnetic coil from plurality of electromagnetic coils 106 is activated, it may urge the respective passive magnetic tag from plurality of passive magnetic tags 104 to move toward the respective electromagnetic coil from plurality of electromagnetic coils 106.

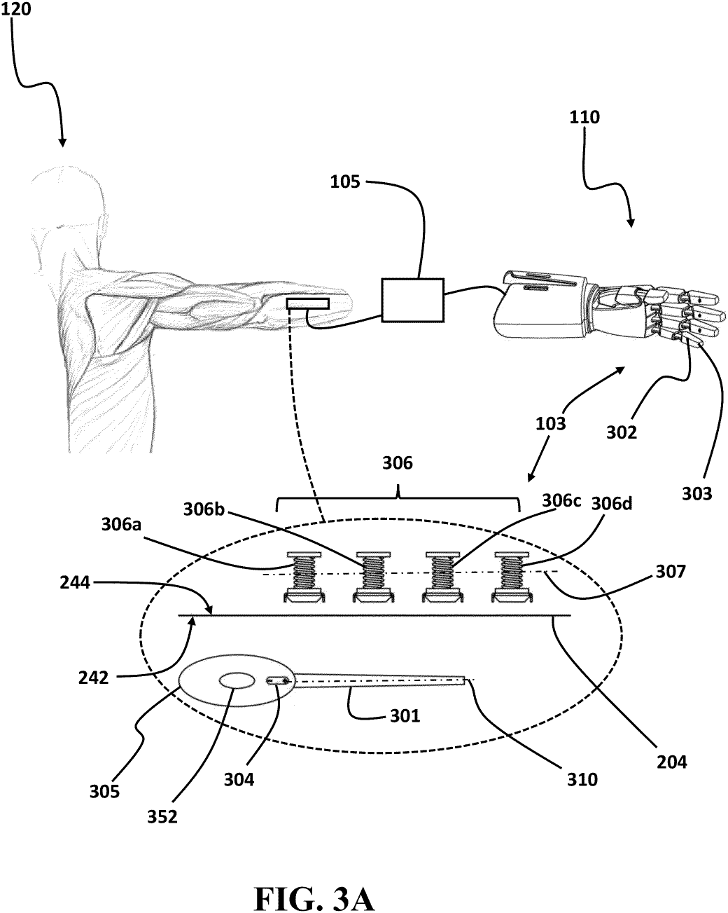

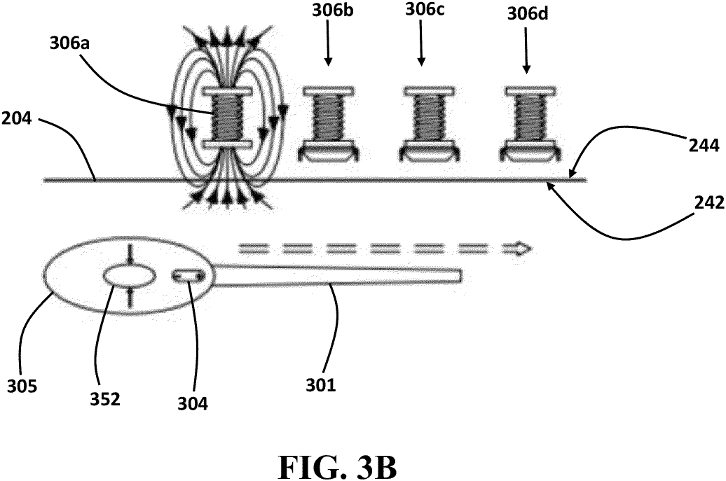

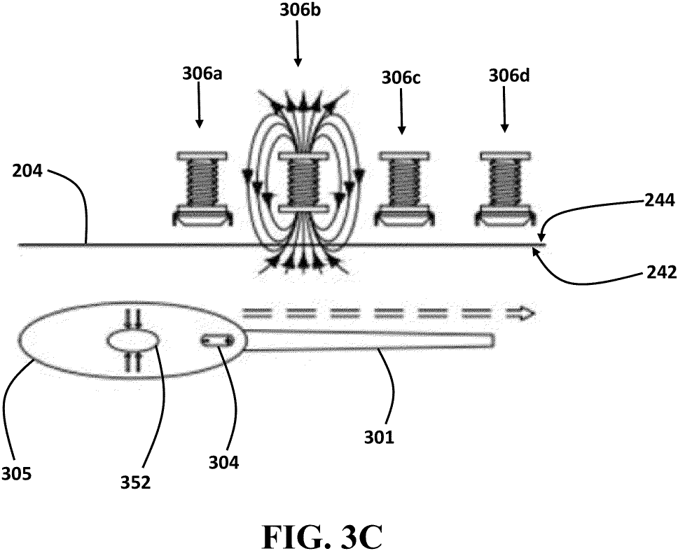

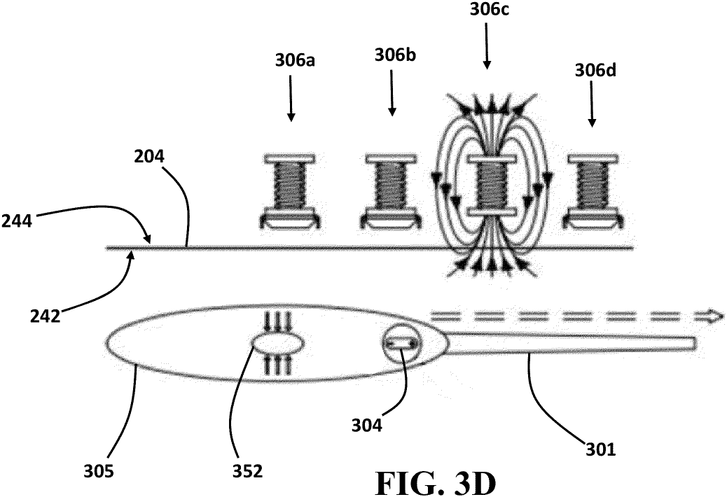

FIG. 3A shows a proprioception unit 103 of a haptic perception system with hand prosthesis 110 of an amputee 120, consistent with one or more exemplary embodiments of the present disclosure. In an exemplary embodiment, proprioception unit 103 may include a strain gauge 302. In an exemplary embodiment, strain gauge 302 may be attached to a finger 303 of hand prosthesis 110. In an exemplary embodiment, strain gauge 302 may be configured to measure strain in finger 303 of hand prosthesis 110. In an exemplary embodiment, it may be understood that a strain gauge is a sensor whose measured electrical resistance varies with changes in strain. Strain is the deformation or displacement of material that results from an applied stress. Stress is the force applied to a material, divided by the material’s cross-sectional area. Then, when the strain in finger 303 of hand prosthesis 110 changes, strain gauge’s measured electrical resistance varies and therefore, the strain gauge may measure strain in finger 303. In an exemplary embodiment, proprioception unit 103 may further include a muscle magnetic tag 304. In an exemplary embodiment, muscle magnetic tag 304 may be embedded in a muscle 305 of residual limb 122. In an exemplary embodiment, muscle magnetic tag 304 may have a cylindrical shape with a size proportional to muscle 305 in which it is inserted. In an exemplary embodiment, muscle magnetic tag 304 may be placed at a musculotendinous junction between muscle 305 and tendon 301. In an exemplary embodiment, proprioception unit 103 may further include a row of electromagnetic coils 306. In an exemplary embodiment, row of electromagnetic coils 306 may be attached onto skin 204 of residual limb 122 of amputee 120. In an exemplary embodiment, row of electromagnetic coils 306 may be arranged along a first axis 307. In an exemplary embodiment, first axis 307 may be parallel to a main longitudinal axis 310 of a tendon 301 of residual limb 122 of amputee 120. In an exemplary embodiment, tendon 301 may be directly connected to muscle 305.

In an exemplary embodiment, row of electromagnetic coils 306 may include a second electromagnetic coil 306a, a third electromagnetic coil 306b, a fourth electromagnetic coil 306c, and a fifth electromagnetic coil 306d. In an exemplary embodiment, each electromagnetic coil from row of electromagnetic coils 306 may be configured to urge muscle magnetic tag 304 to move toward the each electromagnetic coil when the each electromagnetic coil is activated. In an exemplary embodiment, when the each electromagnetic coil from row of electromagnetic coils 306 is activated, a magnetic field may be created around the each electromagnetic coil from row of electromagnetic coils 306 that may urge muscle magnetic tag 304 to move toward the each electromagnetic coil. For example, when second electromagnetic coil 306a is activated, second electromagnetic coil 306a may urge muscle magnetic tag 304 to move toward second electromagnetic coil 306a. In an exemplary embodiment, when third electromagnetic coil 306b is activated, muscle magnetic tag 304 may move further along main longitudinal axis 310 of tendon 301 in comparison with a scenario in which second electromagnetic coil 306a is activated. In an exemplary embodiment, when fourth electromagnetic coil 306c is activated, muscle magnetic tag 304 may move further along main longitudinal axis 310 of tendon 301 in comparison with a scenario in which third electromagnetic coil 306b is activated. In an exemplary embodiment, when fifth electromagnetic coil 306d is activated, muscle magnetic tag 304 may move further along main longitudinal axis 310 of tendon 301 in comparison with a scenario in which fourth electromagnetic coil 306c is activated.

In an exemplary embodiment, one or more processors 105 may further be configured to capture a second set of data from strain gauge 302. In an exemplary embodiment, one or more processors 105 may further be configured to activate one electromagnetic coil from row of electromagnetic coils 306 by sending a second activation signal to the one electromagnetic coil from row of electromagnetic coils 306.

In an exemplary embodiment, when strain of finger 303 is less than a first predetermined amount, one or more processors 105 may send the second activation signal to second electromagnetic coil 306a and, thereby, muscle magnetic tag 304 may move by a first distance toward row of electromagnetic coils 306. In an exemplary embodiment, when strain of finger 303 is greater than the first predetermined amount and less than a second predetermined amount, one or more processors 105 may send the second activation signal to third electromagnetic coil 306b and, thereby, muscle magnetic tag 304 may move by a second distance toward row of electromagnetic coils 306. In an exemplary embodiment, when strain of finger 303 is greater than the second predetermined amount and less than a third predetermined amount, one or more processors 105 may send the second activation signal to fourth electromagnetic coil 306c and, thereby, muscle magnetic tag 304 may move by a third distance toward row of electromagnetic coils 306. In an exemplary embodiment, when strain of finger 303 is greater than the third predetermined amount, one or more processors 105 may send the second activation signal to fifth electromagnetic coil 306d and, thereby, muscle magnetic tag 304 may move by a fourth distance toward row of electromagnetic coils 306. In an exemplary embodiment, the fourth distance may be greater than the third distance which may be greater than the second distance which may be greater than the first distance.

In an exemplary embodiment, when muscle magnetic tag 304 moves toward row of electromagnetic coils 306, muscle 305 may be pulled and/or pushed and, thereby, a muscle mechanoreceptor 352 may be stimulated. For purpose of reference, it should be understood that when muscle mechanoreceptor 352 is stimulated, an electrical signal may be generated and sent to the central nervous system of the amputee which may induce proprioceptive sense.

FIG. 3B shows row of electromagnetic coils 306 and muscle magnetic tag 304 of proprioception unit 103, in a scenario in which second electromagnetic coil 306a is activated, consistent with one or more exemplary embodiments of the present disclosure. FIG. 3C shows row of electromagnetic coils 306 and muscle magnetic tag 304, in a scenario in which third electromagnetic coil 306b is activated, consistent with one or more exemplary embodiments of the present disclosure. FIG. 3D shows row of electromagnetic coils 306 and muscle magnetic tag 304, in a scenario in which fourth electromagnetic coil 306c is activated, consistent with one or more exemplary embodiments of the present disclosure.

In an exemplary embodiment, a system which may be similar to an exemplary haptic perception system in structure and functionality, may be used to induce stereognostic and proprioceptive senses for any artificial limb in body. For example, a similar exemplary system may be used for inducing stereognostic and proprioceptive senses for an artificial foot prosthesis. Also, the disclosed haptic perception system 100 may be used for inducing stereognostic and proprioceptive senses in a sensory disrupted limb.

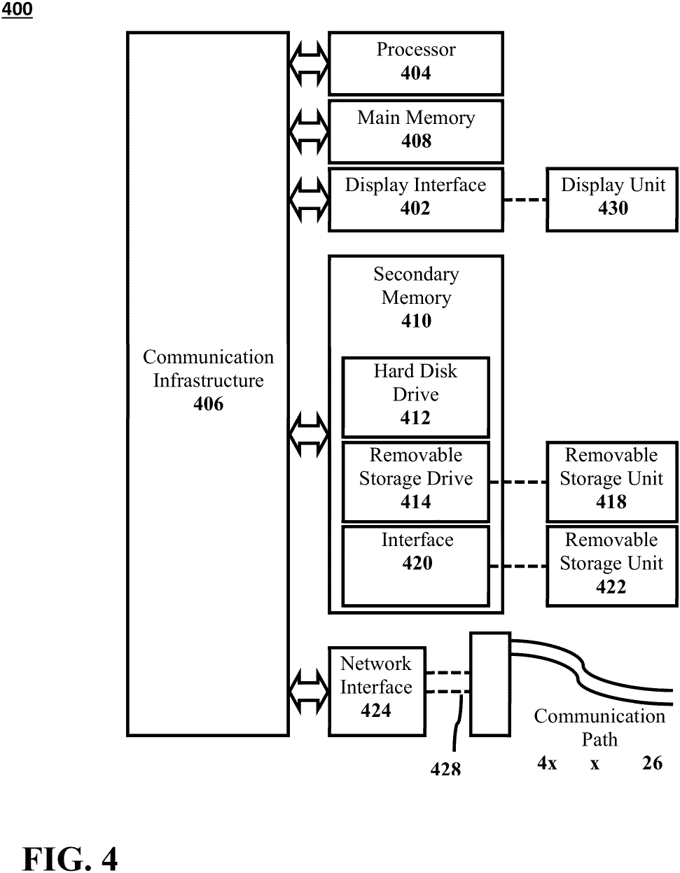

FIG. 4 shows an exemplary embodiment of processing unit 400 in which an exemplary embodiment of the present disclosure, or portions thereof, may be implemented as computer-readable code, consistent with one or more exemplary embodiments of the present disclosure. For example, an exemplary haptic perception system may be implemented in processing unit 400 using hardware, software, firmware, tangible computer readable media having instructions stored thereon, or a combination thereof and may be implemented in one or more computer systems or other processing systems.

If programmable logic is used, such logic may execute on a commercially available processing platform or a special purpose device. One of ordinary skill in the art may appreciate that an exemplary embodiment of the disclosed subject matter can be practiced with various computer system configurations, including multi-core multiprocessor systems, minicomputers, mainframe computers, computers linked or clustered with distributed functions, as well as microcontrollers, pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An exemplary embodiment of the present disclosure is described in terms of this example processing unit 400. After reading this description, it will become apparent to a person skilled in the relevant art how to implement the present disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 404 may be a special purpose or a general-purpose processor device. As will be appreciated by persons skilled in the relevant art, processor device 404 may also be a single processor in a multi-core/multiprocessor system, such system operating alone, or in a cluster of computing devices operating in a cluster or server farm. In an exemplary embodiment, processor device 404 may be connected to a communication infrastructure 406, for example, a bus, message queue, network, or multi-core message-passing scheme.

In an exemplary embodiment, processing unit 400 may also include a main memory 408, for example, random access memory (RAM), and may also include a secondary memory 410. In an exemplary embodiment, secondary memory 410 may include a hard disk drive 412, and a removable storage drive 414. In an exemplary embodiment, removable storage drive 414 may include a floppy disk drive, a magnetic tape drive, an optical disk drive, a flash memory, or the like. In addition, removable storage drive 414 may read from and/or write to a removable storage unit 418 in a well-known manner. In an exemplary embodiment, removable storage unit 418 may include a floppy disk, magnetic tape, optical disk, etc., which may be read by and written to by removable storage drive 414. As will be appreciated by persons skilled in the relevant art, removable storage unit 418 may include a computer usable storage medium having stored therein computer software and/or data.

In alternative implementations, secondary memory 410 may include other similar means for allowing computer programs or other instructions to be loaded into one or more processors 105. Such means may include, for example, a removable storage unit 422 and an interface 420. Examples of such means may include a program cartridge and cartridge interface (such as that found in video game devices), a removable memory chip (such as an EPROM, or PROM) and associated socket, and other removable storage units 422 and interfaces 420 which allow software and data to be transferred from removable storage unit 422 to processing unit 400.

In an exemplary embodiment, processing unit 400 may also include a communications interface 424. Communications interface 424 may allow software and data to be transferred between processing unit 400 and external devices. In an exemplary embodiment, communications interface 424 may include a modem, a network interface (such as an Ethernet card), a communications port, a PCMCIA slot and card, or the like. Software and data transferred via communications interface 424 may be in the form of signals, which may be electronic, electromagnetic, optical, or other signals capable of being received by communications interface 424. These signals may be provided to communications interface 424 via a communications path 426. In an exemplary embodiment, communications path 426 may carry signals and may be implemented using wire or cable, fiber optics, a phone line, a cellular phone link, an RF link or other communications channels.

In this document, the terms “computer program medium” and “computer usable medium” are used to generally refer to media such as removable storage unit 418, removable storage unit 422, and a hard disk installed in hard disk drive 412. Computer program medium and computer usable medium may also refer to memories, such as main memory 408 and secondary memory 410, which may be memory semiconductors (e.g. DRAMs, etc.).

In some exemplary embodiment, computer programs (also called computer control logic) may be stored in main memory 408 and/or secondary memory 410. Computer programs may also be received via communications interface 424. Such computer programs, when executed, enable processing unit 400 to implement the present disclosure as discussed herein. In particular, the computer programs, when executed, may enable processor device 404 to implement the processes of the present disclosure. Accordingly, such computer programs represent controllers of processing unit 400. Where the present disclosure is implemented using software, the software may be stored in a computer program product and loaded into processing unit 400 using removable storage drive 414, interface 420, and hard disk drive 412, or communications interface 424.

Embodiments of the present disclosure may also be directed to computer program products including software stored on any computer useable medium. Such software, when executed in one or more data processing devices, causes a data processing device(s) to operate as described herein. An exemplary embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Ends 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective spaces of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A haptic perception system for a hand prosthesis of an amputee, the haptic perception system comprising:
a stereognosis unit comprising:
a plurality of pressure sensors attached onto an external surface of the hand prosthesis, a first pressure sensor from the plurality of pressure sensors configured to measure a pressure applied to the first pressure sensor;
a plurality of passive magnetic tags, the plurality of passive magnetic tags configured to be embedded in a hypoderm tissue of a residual limb of the amputee, a first passive magnetic tag from the plurality of passive magnetic tags associated with the first pressure sensor; and
a plurality of electromagnetic coils, the plurality of electromagnetic coils configured to be attached onto a skin of the residual limb of the amputee, a first electromagnetic coil from the plurality of electromagnetic coils associated with the first pressure sensor, the first electromagnetic coil configured to be placed above the first passive magnetic tag from the plurality of passive magnetic tags, the first electromagnetic coil configured to urge the first passive magnetic tag to move toward the first electromagnetic coil responsive to activation of the first electromagnetic coil;
a proprioception unit, comprising:
a strain gauge attached to a finger of the hand prosthesis, the strain gauge configured to measure strain in the finger of the hand prosthesis;
a muscle magnetic tag, the muscle magnetic tag configured to be embedded in a muscle of the residual limb, the muscle magnetic tag associated with the strain gauge; and
a row of electromagnetic coils, the row of electromagnetic coils configured to be attached onto the skin of the residual limb of the amputee, the row of electromagnetic coils configured to be arranged along a first axis, the first axis parallel to a main longitudinal axis of a tendon of the residual limb of the amputee, the tendon of the residual limb of the amputee directly connected to the muscle of the residual limb, each electromagnetic coil from the row of electromagnetic coils configured to urge the muscle magnetic tag to move toward the each electromagnetic coil responsive to activation of the each electromagnetic coil; and
one or more processors configured to:
capture a first set of data from the first pressure sensor, the first set of data associated with a pressure applied to the first pressure sensor;
detect the pressure applied to the first pressure sensor being higher than a threshold based on the first set of data;
activate the first electromagnetic coil by sending a first activation signal to the first electromagnetic coil responsive to the pressure applied to the pressure sensor being higher than the threshold;
capture a second set of data from the strain gauge, the second set of data associated with the strain of the finger of the hand prosthesis; and
activate one electromagnetic coil from the row of electromagnetic coils by sending a second activation signal to the one electromagnetic coil from the row of electromagnetic coils based on the second set of data; wherein:
responsive to activation of the first electromagnetic coil, the first passive magnetic tag is configured to stimulate pressure sensory receptors present in the hypoderm tissue of the residual limb by the first passive magnetic tag is configured to move toward the first electromagnetic coil and;
each pressure sensor from the plurality of pressure sensors is configured to measure a pressure applied to the each pressure sensor;
each electromagnetic coil from the plurality of electromagnetic coils is configured to be placed above a respective passive magnetic tag from the plurality of passive magnetic tags.
each electromagnetic coil from the plurality of electromagnetic coils is configured to urge the respective passive magnetic tag to move toward the each electromagnetic coil responsive to activation of the each electromagnetic coil;
first electromagnetic coil is attached onto a hollow base;
the hollow base is configured to be attached onto the skin of the residual limb;
the hollow base is configured to prevent a direct contact between the first electromagnetic oil and the skin of the residual limb;
the first passive magnetic tag is embedded in a capsule;
the capsule is covered by a hydroxyapatite layer;
a distance between two adjacent passive magnetic tags of the plurality of passive magnetic tags is less than 7 mm;
a distance between two adjacent electromagnetic coils of the plurality of electromagnetic coils is less than 7 mm;
the row of electromagnetic coils comprises four electromagnetic coils comprising a second electromagnetic coil, a third electromagnetic coil, a fourth electromagnetic coil, and a fifth electromagnetic coil; and
the one or more processors are further configured to:
activate the second electromagnetic coil from the row of electromagnetic coils by sending the second activation signal to the second electromagnetic coil from the row of electromagnetic coils responsive to the strain of the finger of the hand prosthesis being less than a first predetermined amount;
activate the third electromagnetic coil from the row of electromagnetic coils by sending the second activation signal to the third electromagnetic coil from the row of electromagnetic coils responsive to the strain of the finger of the hand prosthesis being greater than the first predetermined amount and less than a second predetermined amount;
activate the fourth electromagnetic coil from the row of electromagnetic coils by sending the second activation signal to the fourth electromagnetic coil from the row of electromagnetic coils responsive to the strain of the finger of the hand prosthesis being greater than the second predetermined amount and less than a third predetermined amount; and
activate the fifth electromagnetic coil from the row of electromagnetic coils by sending the second activation signal to the fifth electromagnetic coil from the row of electromagnetic coils responsive to the strain of the finger of the hand prosthesis being greater than the third predetermined amount.

2. A haptic perception system for a hand prosthesis of an amputee, the haptic perception system comprising:
a stereognosis unit, comprising:
a plurality of pressure sensors attached onto an external surface of the hand prosthesis, a first pressure sensor from the plurality of pressure sensors configured to measure a pressure applied to the first pressure sensor;
a plurality of passive magnetic tags, the plurality of passive magnetic tags configured to be embedded in a hypoderm tissue of a residual limb of the amputee, a first passive magnetic tag from the plurality of passive magnetic tags associated with the first pressure sensor; and
a plurality of electromagnetic coils, the plurality of electromagnetic coils configured to be attached onto a skin of the residual limb of the amputee, a first electromagnetic coil from the plurality of electromagnetic coils associated with the first pressure sensor, the first electromagnetic coil configured to be placed above the first passive magnetic tag from the plurality of passive magnetic tags, the first electromagnetic coil configured to urge the first passive magnetic tag to move toward the first electromagnetic coil responsive to activation of the first electromagnetic coil; and
one or more processors configured to:
capture a first set of data from the first pressure sensor, the first set of data associated with a pressure applied to the first pressure sensor;
detect the pressure applied to the first pressure sensor being higher than a threshold based on the first set of data; and
activate the first electromagnetic coil by sending a first activation signal to the first electromagnetic coil responsive to the pressure applied to the pressure sensor being higher than the threshold,
wherein responsive to activation of the first electromagnetic coil, the first passive magnetic tag is configured to stimulate pressure sensory receptors present in the hypoderm tissue of the residual limb by the first passive magnetic tag moving toward the first electromagnetic coil.

3. The haptic perception system of claim 2, further comprising:
a proprioception unit, comprising:
a strain gauge attached to a finger of the hand prosthesis, the strain gauge configured to measure strain in the finger of the hand prosthesis;
a muscle magnetic tag, the muscle magnetic tag configured to be embedded in a muscle of the residual limb, the muscle magnetic tag associated with the strain gauge; and
a row of electromagnetic coils, the row of electromagnetic coils configured to be attached onto the skin of the residual limb of the amputee, the row of electromagnetic coils configured to be arranged along a first axis, the first axis parallel to a main longitudinal axis of a tendon of the residual limb of the amputee, the tendon of the residual limb of the amputee directly connected to the muscle of the residual limb, each electromagnetic coil from the row of electromagnetic coils configured to urge the muscle magnetic tag to move toward the each electromagnetic coil responsive to activation of the each electromagnetic coil; wherein:
the one or more processors are further configured to:
capture a second set of data from the strain gauge, the second set of data associated with the strain of the finger of the hand prosthesis; and
activate one electromagnetic coil from the row of electromagnetic coils by sending a second activation signal to the one electromagnetic coil from the row of electromagnetic coils based on the second set of data.

4. The haptic perception system of claim 3, wherein:
each pressure sensor from the plurality of pressure sensors is configured to measure a pressure applied to the each pressure sensor;
each passive magnetic tag from the plurality of passive magnetic tags is associated with a respective pressure sensor from the plurality of pressure sensors;
each electromagnetic coil from the plurality of electromagnetic coils is associated with a respective pressure sensor from the plurality of pressure sensors;
each electromagnetic coil from the plurality of electromagnetic coils is configured to be placed above a respective passive magnetic tag from the plurality of passive magnetic tags;
each electromagnetic coil from the plurality of electromagnetic coils is configured to urge the respective passive magnetic tag to move toward the each electromagnetic coil responsive to activation of the each electromagnetic coil.

5. The haptic perception system of claim 4, wherein:
the first electromagnetic coil is attached onto a hollow base;
the hollow base is configured to be attached onto the skin of the residual limb; and
the hollow base is configured to prevent a direct contact between the first electromagnetic oil and the skin of the residual limb.

6. The haptic perception system of claim 5, wherein:
the first passive magnetic tag is embedded in a capsule; and
the capsule is covered by a hydroxyapatite layer.

7. The haptic perception system of claim 6, wherein:
a distance between two adjacent passive magnetic tags of the plurality of passive magnetic tags is less than 7 mm; and
a distance between two adjacent electromagnetic coils of the plurality of electromagnetic coils is less than 7 mm.

8. The haptic perception system of claim 7, wherein the row of electromagnetic coils comprises four electromagnetic coils comprising a second electromagnetic coil, a third electromagnetic coil, a fourth electromagnetic coil, and a fifth electromagnetic coil.

9. The haptic perception system of claim 8, wherein the one or more processors are further configured to:
activate the second electromagnetic coil from the row of electromagnetic coils by sending the second activation signal to the second electromagnetic coil from the row of electromagnetic coils responsive to the strain of the finger of the hand prosthesis being less than a first predetermined amount;
activate the third electromagnetic coil from the row of electromagnetic coils by sending the second activation signal to the third electromagnetic coil from the row of electromagnetic coils responsive to the strain of the finger of the hand prosthesis being greater than the first predetermined amount and less than a second predetermined amount;
activate the fourth electromagnetic coil from the row of electromagnetic coils by sending the second activation signal to the fourth electromagnetic coil from the row of electromagnetic coils responsive to the strain of the finger of the hand prosthesis being greater than the second predetermined amount and less than a third predetermined amount; and
activate the fifth electromagnetic coil from the row of electromagnetic coils by sending the second activation signal to the fifth electromagnetic coil from the row of electromagnetic coils responsive to the strain of the finger of the hand prosthesis being greater than the third predetermined amount.

10. The haptic perception system of claim 9, wherein:
the plurality of passive magnetic tags are attached onto a sheet; and
the sheet is configured to be embedded in the hypoderm tissue of the residual limb of the amputee.

11. The haptic perception system of claim 10, wherein the muscle magnetic tag is configured to be placed at a musculotendinous junction between the muscle and the tendon.

12. The haptic perception system of claim 10, wherein the pressure sensory receptors comprises Meissner’s and/or Pacinian corpuscles.

13. A haptic perception system for a hand prosthesis of an amputee, the haptic perception system comprising:
a pressure sensor attached to the hand prosthesis, the pressure sensor configured to measure a pressure applied to the hand prosthesis;
a passive magnetic tag configured to be embedded in a hypoderm tissue of a residual limb of the amputee;
an electromagnetic coil configured to be attached onto a skin of the residual limb of the amputee, the electromagnetic coil configured to urge the passive magnetic tag to move toward the first electromagnetic coil responsive to activation of the first electromagnetic coil; and
one or more processors configured to activate the electromagnetic coil responsive to the pressure applied to the pressure sensor being higher than a threshold.
